(19) United States
(12) Patent Application Publication
(10) Pub. No.: US 2009/0270699 A1
(43) Pub. Date: Oct. 29, 2009

(54) Device for Determining Physiological Variables

(76) Inventors: Bernd Scholler, Karlsruhe (DE); Thomas Magin, Schifferstadt (DE); Klaus Forstner, Asperg (DE)

Correspondence Address: FRIEDRICH KUEFFNER, 317 MADISON AVENUE, SUITE 910, NEW YORK, NY, 10017, US

(21) Appl. No.: 12/084,994

(22) PCT Filed: Nov. 2, 2006
(86) PCT No.: PCT/DE2006/001934

§ 371 Date: Mar. 2, 2009

(30) Foreign Application Priority Data
Nov. 15, 2005 (DE) 102005054838.5

(51) Int. Cl. A61B 5/1455

(52) U.S. Cl. 600/315; 600/310; 600/316; 600/322; 600/328

(57) ABSTRACT

The device serves to optically determine physiological variables in perfused tissue. The device comprises a first and a second light source which each emit light radiation of a first or a second predetermined wavelength. The light sources are arranged in such a manner that the light radiation exiting them can penetrate into the perfused tissue. At least one photodetector is used, which is arranged so that it detects the light emitted by the light sources and passing through or backscattered by the perfused tissue. The device also comprises a control unit, which furnishes control signals to the light sources so that the light sources continuously emit light alternately, one or more dark phases can be inserted into this sequence, during which at least one light source does not emit any light. An evaluating device is connected to the output of the photodetector and furnishes, for at least one physiological variable to be measured, a displayable output signal to an interface that can be connected to the evaluating device.

The invention concerns a device for the optical determination of physiological variables in perfused tissue with at least a first and a second light source, which emit light of a first and a second predetermined wavelength, such that the light sources are disposed in such a way that the light they emit is able to penetrate the perfused tissue.

Pulse oximetry allows noninvasive measurement of the oxygen saturation of the blood. In this method, light of two different wavelengths, for example, 660 nm and 905 nm, is passed through a finger. The light is partially absorbed by the blood pulsating in the tissue. The degree of absorption is determined by analysis of the light component emerging from the other side of the irradiated tissue, which allows a direct conclusion about the oxygen saturation of the pulsating and thus arterial blood.

In the usual range of measurement (80-100% saturation), pulse oximetry is quite accurate in comparison to invasively measured arterial oxygen saturation. The limits of pulse oximetry are reached, for example, when an intoxication is present, e.g., carbon monoxide intoxication, or in cases of drug-induced toxic methemoglobinemia. In these cases, pulse oximetry yields false-high oxygen saturation values, which can have dangerous consequences. Moreover, pulse oximetry is unsuitable for determining the oxygen concentration (caO2). To evaluate a patient's oxygen supply, additional information about the hemoglobin concentration is needed.

There is a need for a fast and highly accurate, noninvasive determination of several physiological variables (pV's), which in their totality make it possible to evaluate the oxygen supply of a patient.

In accordance with the invention, this goal is achieved by the combination of the following features:

(a) at least one photodetector (PD), which is disposed in such a way that it detects the light that is emitted by the light sources and transmitted and/or backscattered by the perfused tissue;

(b) a control unit, which supplies control signals to the light sources in such a way that the light sources continuously emit light in alternation with each other, such that one or more dark phases can be inserted in this sequence, in which at least one of the light sources emits no light;

(c) an evaluation unit, which is connected with the output of the photodetector (PD),

(d) such that, for at least one pV to be measured, the evaluation unit supplies a displayable output signal to an interface that can be connected to the evaluation unit.

The invention proposes a device and a method which allow a noninvasive determination of several physiological variables (pV's) selected from the group comprising temperature, pulse rate, pH, concentration of hemoglobin (cHb), oxyhemoglobin (HbO2), deoxyhemoglobin (HbDe), carboxyhemoglobin (HbCO), methemoglobin (cMetHb), sulfmethemoglobin (HbSulf), bilirubin, glucose, bile pigments, SaO2, SaCO, SpO2, CaO2, and SPCO. Preferably, the device of the invention and the method of the invention allow a noninvasive determination of several physiological variables (pV's) by one instrument.

More precisely, the invention concerns a device which noninvasively records, compensates, and processes a physiological variable (pV) in order to supply an output signal that represents the value of the pV at the time of the measurement.

The device of the invention involves the use of a noninvasive method. To determine measured values, one or more light sources are placed on a part of the body. One or more analyzing photocells are provided some distance from the light sources to detect light attenuation and/or receive a scattered light component.

The source of electromagnetic radiation may be, for example, one or more laser diodes and/or one or more white light sources and/or one or more LED's.

The electromagnetic radiation is selected from one or more ranges of 150 nm±15%, 400 nm±15%, 460 nm±15%, 480 nm±15%, 520 nm±15%, 550 nm±15%, 560 nm±15%, 606 nm±15%, 617 nm±15%, 620 nm±15%, 630 nm±15%, 650 nm±15%, 660 nm±15%, 705 nm±15%, 710 nm±15%, 720 nm±15%, 805 nm±15%, 810 nm±15%, 880 nm±15%, 905 nm±15%, 910 nm±15%, 950 nm±15%, 980 nm±15%, 980 nm±15%, 1050 nm±15%, 1200 nm±15%, 1310 nm±15%, 1380 nm±15%, 1450 nm±15%, 1600 nm±15%, 1800 nm±15%, 2100 nm±15%, 2800 nm±15%.

The electromagnetic waves are passed through a living and/or dead medium, preferably animal and/or human tissue.

The transmitted and/or reflected component of the electromagnetic waves is detected by a receiving system, which preferably takes the form of one or more photodetectors. The receiving system is capable of detecting different wavelengths essentially simultaneously. The receiving system is also capable of recording and/or storing and/or relaying the detected electromagnetic waves, for example, in the form of at least one electric pulse, preferably as a current and/or voltage signal.

The signal is subjected to signal conditioning by an evaluation unit. Regardless of the original wavelength, the one or more signals are further processed by active and/or passive electronic components. An adjustment according to frequency and amplitude is preferably carried out. It is especially preferred that an adjustment of the ratio of the AC to DC component and/or their level be carried out by filters, noise suppression, capacitors, amplifiers, high-pass filters, and logic flip-flops. The result is that the output of the evaluation unit is preferably a processed AC component of the output signal.

The processed signal is digitalized by an a/d converter with a high bit width and resolution. An a/d converter of at least 12 bits is preferably used for this purpose.

Digital signals that are representative of at least two different wavelengths of the originally radiated electromagnetic radiation are analyzed by at least one CPU. An analyzer is preferably provided in a CPU for this purpose. Signal processing is performed in the CPU. At least one memory that can be read out is provided in the CPU for the digital signals.

The following operations are carried out alternatively, sequentially, or simultaneously in the analyzer:

data acquisition and processing

pulse wave characteristic or morphology or parameters derived from them, such as extreme points, derivatives, etc.

absorbances are determined (computed or read out)

artifact cleaning of internal and external artifacts (movement, rearrangement, perfusion, . . . )

parallel series of measured values are combined and back-calculated to a new result.

computation and conditioning of an analog or digital signal for controlling other modules or instruments.

The result is that the CPU supplies data that is representative of at least one pV of the irradiated medium. The output of the CPU is preferably coupled with a controller, which is coupled with a d/a converter. The controller feeds back to the source and controls the intensity of the radiation being emitted by the source in such a way that the radiation intensity detected by the receiving system and relayed to the a/d converter is always in a preferred resolution range.

Alternatively, if a self-adaptive system is chosen, it is possible to dispense entirely with the feedback to the source (and the controller).

Artifact cleaning is preferably carried out by a CPU, which further processes the output signal of the evaluation unit in the time domain (for example, by polynomial functions) or the Laplace domain (for example, by Fourier transform or wavelets). The functions are selected in such a way that they are adapted to the possible artifact properties.

With the use of this compensation method, the pV's are typically determined to an accuracy of at least 5%, and preferably 2%, over the range of measurement of the given pV.

For example, the range of measurement for the concentration of hemoglobin cHb is typically 5-20 g/dL, and the range of normal values is 14-18 g/dL for men and 12-16 g/dL for women.

In general, at Hct 24%/cHb 8 g/dL, even with normal O2 consumption and under otherwise favorable conditions, the compensation mechanisms of the body are being fully utilized. Therefore, in accordance with the invention, it is proposed that the cHb be noninvasively determined with an accuracy of 1.5-2.0 g/dL, and preferably 1 g/dL, such that the measured value for cHb is available preferably in less than 1 minute and especially in less than 10 seconds. In accordance with the invention, to determine cHb, at least four wavelengths from the range of 600 nm to 1500 nm are used.

The range of values for bilirubin is typically 0.1 to 5.0 mg/dL. Therefore, in accordance with the invention, it is proposed that bilirubin be noninvasively determined with an accuracy of 0.1-1.0 g/dL, and preferably 0.5 g/dL, such that the measured value for bilirubin is available preferably in less than 1 minute, and especially in less than 10 seconds. In accordance with the invention, to determine bilirubin, at least two wavelengths selected from the range of 400 nm to 2000 nm are used.

The range of values for blood oxygen O2 is typically 40% to 100% saturation, and physiological and/or pathophysiological fluctuations can occur quite rapidly.

Therefore, in accordance with the invention, it is proposed that SaO2 be noninvasively determined with an accuracy of at least 5%, and preferably at least 2%, such that the measured value for oxygen is available in less than 1 minute, and especially in less than 5 seconds. In accordance with the invention, to determine SaO2, at least two wavelengths selected from the range of 600 nm to 1000 nm are used.

The range of values for the concentration of carbon monoxide SaCO in the blood is typically 0% to 40% saturation, and changes can occur quite rapidly.

Therefore, in accordance with the invention, it is proposed that SaCO be noninvasively determined with an accuracy of at least 5%, and preferably at least 2%, such that the measured value for the fraction of carbon monoxide SaCO in the blood is determined in less than 100 seconds, preferably in less than 20 second, and especially in less than 5 seconds. In accordance with the invention, to determine SaCO, at least two wavelengths from the range of 600 nm to 1000 nm are used.

The speed and accuracy of the determination of the pV depend on the amount of electric power that is available and the time required for the calculation of the test signals. Especially in the case of portable instruments that are operated with batteries, the available electric power is a limiting factor.

Errors in the performance of the device can be complex and can be a nonlinear function of many variables. The pV contributes directly to the error, while secondary process variables (which affect the measurement of the primary process variables) enter into the error indirectly. Since the need for accuracy is increasing, the contributions of the secondary variables are becoming more and more important.

In addition to the problems of software complexity and computational complexity, the power consumption, for example, of the CPU, is critical, because the total operating power or voltage supply is provided over the same lines that are used for communication. In addition, some intrinsically reliable areas limit the power available for the device. The limited current supply not only limits the number and the complexity of the calculations but also affects the functionality that can be realized in the device.

Especially the sensor, the CPU, and the signal processing require a great deal of power. Therefore, there is a need for an exact method for determining pV's, which is computationally simple and requires a small number of stored property constants, so that a smaller amount of power is consumed, and a greater amount of power is available for additional functionality and increased updating speeds or rates and/or for a faster CPU.

Therefore, in accordance with the invention, it is proposed that all functions of the device be combined on preferably one or two printed circuit boards in order to keep the power requirement low. In accordance with the invention, it is also proposed that a color display not be used and that a black-and-white display or gray-scale display be used instead.

In accordance with the invention, to increase the speed, it is proposed that the pulse wave be quickly identified by presetting the a/d converter to the bandwidth of the pulsation signal to be expected.

In accordance with the invention, to increase the speed, it is further proposed that a 24-bit a/d converter be used for the further processing of the signal received from the PD.

In accordance with the invention, this makes it possible to identify the pulse wave in 20 ms at the longest.

In accordance with the invention, to increase the speed, it is also proposed that the transmission of the electromagnetic wavelengths be used as an automatic control parameter.

Specific embodiments of the invention are schematically illustrated in the drawings.

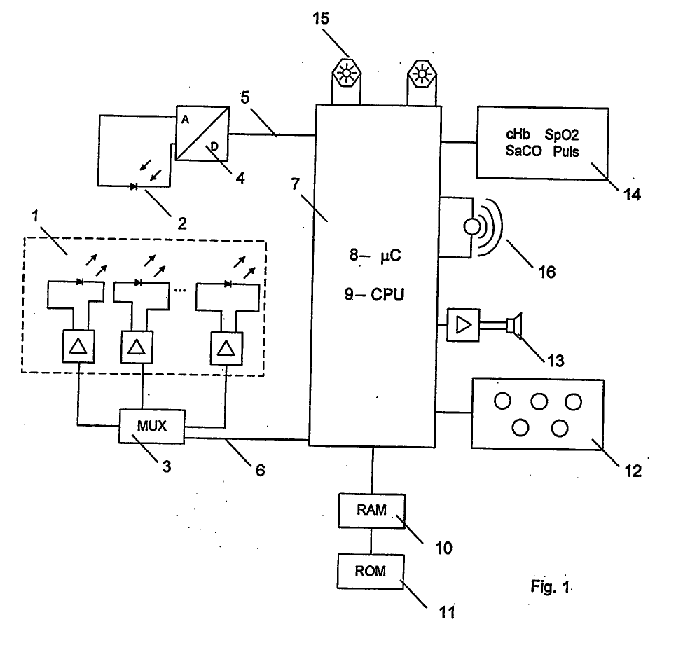

FIG. 1 is a schematic representation of the circuit diagram of the device.

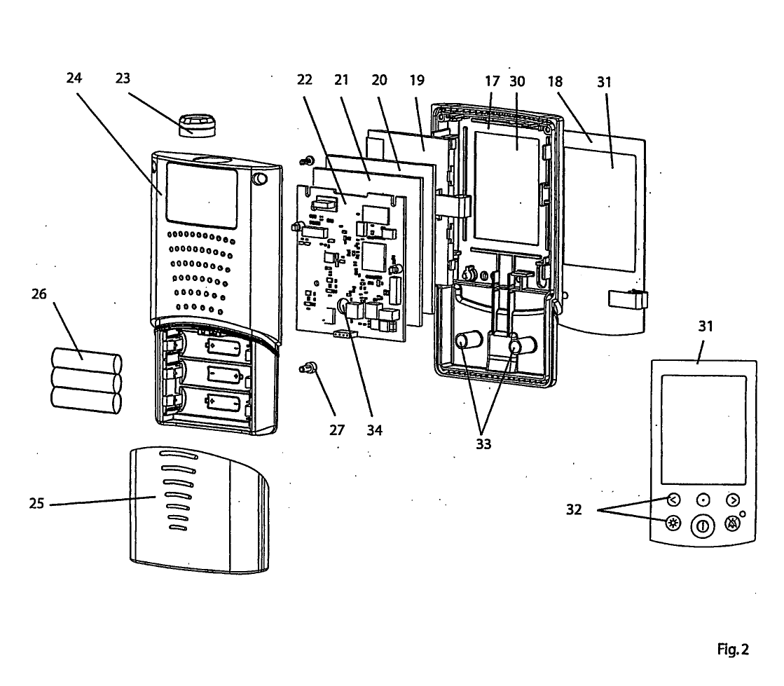

FIG. 2 is a schematic representation of the components of the device.

A device of the invention according to FIG. 1 has a transmitter unit 1, which has at least one light-emitting diode LEDa of a first, predetermined nominal wavelength LAMBDAa.

A photodetector PD 2 is positioned opposite the transmitter unit 1. A human and/or animal tissue and/or vessel can be placed between the transmitter unit 1 and the photodetector PD 2 in such a way that the light emitted by the transmitter 1 reaches the photodetector PD 2 after it passes through the tissue and/or vessel. The light intensity received by the PD is converted to an electrical quantity, subjected to analog processing in the device, then subjected to a/d conversion, and further processed digitally.

The light-emitting diodes LEDa, LEDn are connected with a multiplexer MUX 3. The control unit of the multiplexer MUX 3 controls the light-emitting diodes in such a way that in the case, for example, of four connected LED's, all four LED's are alternately turned on and off.

The multiplexer MUX 3 has another terminal 6, which is connected with the evaluation unit 7. The information about the turn-on times of the light-emitting diodes LEDa to LEDn is transmitted over this connection with the evaluation unit 7. The evaluation unit has at least one microcontroller 8 or at least one CPU 9.

The output current of the photodetector PD 2 is supplied to the input of a current-voltage converter 4, which converts the output current of the photodetector into an output voltage. In addition, the analog signal of the PD is digitized by an a/d converter of at least 8 bits and relayed to the evaluation unit 7 by a control element. At least one volatile memory, RAM 10, and one nonvolatile memory, ROM 11, are connected with the evaluation unit 7. The nonvolatile memory 11 is realized, for example, as an EEPROM or flash memory. An algorithm for determining the pV is stored in the nonvolatile memory 11. An input device 12 in the form of a keyboard can be connected to the evaluation unit 7. In addition, various output devices 13, 14, 15, 16 can be connected to the evaluation unit 7. For example, a loudspeaker 13 can be used to generate warning tones or speech outputs, which can inform or guide the user. Luminaires 15 can be used, for example, to generate warning signals and/or status signals. The pV values are displayed by a display 14.

In at least one exemplary operating state of the device of the invention according to FIG. 1, the tissue/vessel is alternately irradiated by the light emitted by the first light-emitting diode LEDa and the light emitted by the other light-emitting diodes LEDn, and the light that passes through the tissue/vessel is detected by the photodetector PD and converted to a photodetector output current. The light-emitting diodes LEDa, LEDn can either be operated with binary control, in which case either no light or light of a preset intensity is emitted at each instant, or, alternatively, the LED's can be operated with an analog signal of a predetermined amplitude. The time interval for controlling the LED's can be set as a function of the pulse-wave phases, for example, every 200 μs.

At two instants t1 and t2, the operation is carried out as follows:

| t1 | t2 |
|---|---|
| 1. wavelength a | wavelength a |
| 2. wavelength b | wavelength b |
| 3. wavelength c | wavelength c |
| 4. wavelength d | wavelength d |
| 5. dark | dark |

In order to convert the current signal with as little noise as possible and with sufficient amplification to a voltage signal that can be used for the further processing in the evaluation unit 7, it is fed to the current-voltage converter 4 and the a/d converter. The evaluation unit 7 uses the voltage signal to determine the course of the spectral absorption of the tissue/vessel at the wavelengths, which are defined by the LED, of the first and additional light-emitting diodes LEDa, LEDn, and then uses these spectral absorption values to determine, by conditioning and/or further processing and/or linkage, the given pV of interest, for example the absolute or relative hemoglobin concentration Hb, the COHb, the oxygen saturation SaO2, CaO2, or the heart rate. The measured values for the pV for each wavelength are stored in the volatile memory 10 and/or the nonvolatile memory 11. The measured values are then read out again by the evaluation unit 7 with the aid of the microcontroller 8 and analyzed in the CPU 9 by means of the algorithm stored in the ROM 11.

In this connection, digitized data that represent the attenuation and/or scattering of electromagnetic radiation by a tissue/vessel are processed in a central unit under program control, such that a control unit receives the commands of a program from a memory and carries out operations according to the program instructions by means of a computing unit, which consists of at least one arithmetic-logic unit.

Absolute and/or relative measured values are determined as the result for the desired pV. Depending on limiting values and/or preset values, which can be defined via the keyboard 12, the pV results are output electronically, optically 14, 15, and/or acoustically 13. To this end, the data that represent the pV are conditioned for an interface and supplied to an interface. Preferably, a protocol is provided through an interface. For example, a voltage and/or a current that is essentially proportional to the pV is provided at the interface. For example, a digitized value that represents the pV can be made available in a TCP/IP protocol via Ethernet.

For example, an SaO2 value can be supplied to a UART interface via a proprietary protocol.

Another aspect of the invention, which is illustrated in FIG. 2, involves a small, handy, portable device that allows a user to make noninvasive determinations of several pV's. The device consists of a plastic upper housing part 17, which has an opening for a display 30 and openings for operating buttons. A control panel 18 with buttons is inserted in the upper housing part 17. The control panel has openings for a display 31 and operating buttons 32. The display 19 can be joined to the control panel 18 and can be electrically and mechanically connected with the upper part 17 of the housing by the motherboard 20.

A separator 21 mechanically separates the motherboard 20 and the power board 22. Openings 34 for a fastener 27 are provided in the motherboard 20, the lower part 24 of the housing, and the separator 21. Receivers 33 for the fasteners 27 are located on the rear side of the housing upper part 17. There is an adaptable socket 23, which can be electrically and mechanically connected with the motherboard 20 and/or the power board 22. The socket receives a sensor cable. Alternatively, the socket can be equipped as a receiving module for the radio transmission of sensor signals.

The lower part 24 of the housing contains a compartment 28 for a power source 26, for example, batteries. In the assembled state, the lower part 24 and the upper part 17 of the housing are detachably joined by a fastener 27. A cover 25 slides over the compartment 28 for the power supply 26.

The dimensions of the device of the invention are preferably less than 15 cm in length, 8 cm in width, and 5 cm in depth. The volume of the device is preferably less than 600 cm³. To realize small and compact dimensions and nevertheless to ensure that the device can be easily disassembled for servicing and easily reassembled, the device consists of no more than two printed circuit boards 20, 22 and/or fewer than 11 individual parts and/or fewer than three fasteners 27.

In a preferred embodiment, the device of the invention is realized as an original equipment manufacturer (OEM) printed circuit board. The OEM printed circuit board of the invention can be mounted in the vicinity of the printed circuit board of patient monitors by at least one plug contact, which allows detachable mechanical and electronic coupling. In accordance with the invention, it is thus possible, by simply adding the OEM printed circuit board of the invention, to expand the range of functions of patient monitors by the functions that are implemented on the OEM printed circuit board of the invention.

In a preferred embodiment, the device of the invention is designed as an integrated circuit and—due to the low power requirement—with CMOS technology. The printed circuit board is preferably provided with components on both sides. The track width on the printed circuit board is in the range of 0.15 mm±0.5 mm. The distance between tracks is in the range of 0.15 mm±0.5 mm. The operating voltage is preferably in the range of 3 V to 15 V.

The dimensions of the printed circuit board of the invention are preferably less than 100 mm×80 mm, and especially less than 73 mm×39 mm. Preferably, at least one fastening device is provided in the area of the OEM printed circuit board. This fastening device can consist of a drill hole for receiving a screw.

In accordance with the invention, the compact design makes it possible to mount the OEM printed circuit board of the invention in any desired patient monitors. The power supply of the OEM printed circuit board of the invention is provided, for example, via the motherboard of the patient monitor. The data communication between the OEM printed circuit board of the invention and the patient monitor occurs via a definable protocol.

The pV's are determined, for example, by the methods described in DE 103 21 338 A1 and DE 102 13 692 A1. The methods described in DE 103 21 338 A1 and DE 102 13 692 A1 are understood to be part of the present application.

In one embodiment of the invention, the device receives electromagnetic waves, especially light, of at least two different wavelengths and/or of at least two different bands of wavelengths from at least one source, which emits the electromagnetic waves and guides them through a human tissue and/or human vessels to be tested. The transmitted and/or reflected component of the electromagnetic waves is detected by a receiving system, which is suitable for detecting the light signals of different wavelengths in a time interval of less than one second, converting them to current and/or voltage signals that correspond to at least one measured value, and relaying them further. At least one measured value from an evaluation unit is conditioned by signal conditioning, and, independently of the original wavelength, a measured value is further processed by active and/or passive electronic components in order to display it via an interface, for example, on a display.

In another embodiment of the invention, an analog or digital signal is produced from the pV and made available to internal and/or external signal receivers via an interface, such that the measured value of at least one pV is evaluated in a well-defined time, for example, in the range of 10 seconds, and made available for display via an interface.

In another embodiment of the invention, an SaO2 and/or a CaO2 and/or the cHb is determined. Data which represent the SaO2 and/or CaO2 and/or cHb at the time of measurement are determined and processed in such a way that data which represent the current measured values are available to a user and/or are ready for transmission via an interface with a delay of less than 30 seconds, preferably less than 15 seconds, and especially less than 5 seconds, from the time of the emission of a first wavelength until the time of the display.

The invention makes it possible for the first time to use a device to make noninvasive determinations of physiological variables, for example, parameters of oxygen supply in the periphery of the body, and to make them available as information in such a way that, depending on the measured values that are determined, the oxygen supply can be displayed directly and/or indirectly in the time range of less than 5 minutes, preferably less than 2 minutes, and especially less than 30 seconds.

The invention also makes it possible for the first time to use a device to make noninvasive determinations of physiological variables, for example, SaCO, SaO2, cHb, CaO2, and bilirubin, and to make them available as information in such a way that at least two of the pV's can be made available as information in a time interval of less than one minute, and preferably less than 10 seconds.

The invention also makes it possible for the first time to use a device to make noninvasive determinations of physiological variables, for example, SaCO, SaO2, cHb, CaO2, and bilirubin, and to make them available as information in such a way that at least two of the pV's can be made available simultaneously as information by a selection function, for example, on a display.

1. A device for the optical determination of physiological variables (pV's) in perfused tissue with
(a) at least a first light source (LEDa) and a second light source (LEDb), which emit light of a first and a second predetermined wavelength, wherein the light sources are disposed in such a way that the light they emit is able to penetrate the perfused tissue;
(b) at least one photodetector (PD), which is disposed in such a way that it detects the light that is emitted by the light sources and transmitted and/or backscattered by the perfused tissue;
(c) a control unit, which supplies control signals to the light sources in such a way that the light sources continuously emit light in alternation with each other, wherein one or more dark phases can be inserted in this sequence, in which at least one of the light sources emits no light;
(d) an evaluation unit, which is connected with the output of the photodetector (PD),
(e) wherein, for at least one pV to be measured, the evaluation unit supplies a displayable output signal to an interface that can be connected to the evaluation unit.

2. A device in accordance with claim 1, wherein the signals received by the photodetector are processed in a central unit under program control, and a control unit receives the commands of a program from a memory, and operations are carried out according to the program instructions by means of a computing unit, which consists of at least one arithmetic-logic unit.

3. A device in accordance with claim 1, wherein the physiological variable (pV) is selected from the following group: concentration of total hemoglobin cHb, oxyhemoglobin HbO2, deoxyhemoglobin HbDe, carboxyhemoglobin HbCO, methemoglobin HbMet, sulfmethemoglobin HbSulf, bilirubin, glucose, bile pigments, fractional oxygen saturation SaO2, functional oxygen saturation SpO2, oxygen supply CaO2, and carbon monoxide saturation SaCO.

4. A device in accordance with claim 1, wherein the measured value of at least one pV is determined within ten seconds after the recording of the detected electromagnetic waves.

5. A device in accordance with claim 1, wherein within one minute and preferably within thirty seconds after the recording of the detected electromagnetic waves, the measured value is determined, and the signal is ready to be relayed via an interface.

6. A device in accordance with claim 1, wherein the measured values of the carbon monoxide saturation SaCO of the irradiated medium are accurate within two percentage points of the actual saturation in the range of saturation of 0-40%.

7. A device in accordance with claim 1, wherein the measured values of the oxygen saturation SaO2 of the irradiated medium are accurate within two percentage points of the actual saturation in the range of saturation of 0-100%.

8. A device in accordance with claim 1, wherein the measured values of the hemoglobin concentration of the irradiated medium are accurate within 2.0 g/dL of the actual concentration.

9. A device in accordance with claim 1, wherein no more than 60 seconds elapse from the time of the passage of the electromagnetic waves through a tissue/vessel to the output of a value that represents the cHb, such that the concentration cHb of the irradiated tissue/vessel is exact to within 3 g/dL.

10. A device in accordance with claim 1, wherein no more than twenty seconds elapse from the time of the passage of the electromagnetic waves through a tissue/vessel to the output of a value that represents the SaCO, such that the saturation SaCO is exact to within two percentage points (in the range of measurement of 0-40%).

11. A device in accordance with claim 1, wherein a selection can be used to determine which pV is to be determined.

12. A device in accordance with claim 1, wherein a selection can be used to determine how many pV's are to be determined.

13. A device in accordance with claim 1, wherein a selection can be used to determine which pV is to be determined and/or how many pV's are to be determined, and the choice of the wavelengths that are thus suitable for the detection is made and/or can be undertaken on the basis of the selection.

14. A device in accordance with claim 1, wherein at least the concentration of the hemoglobin cHb and the arterial saturation of the blood with oxygen SaO2 can be determined in less than five minutes, and preferably less than two minutes, by using at least four different wavelengths selected from the following group: 400 nm±15%, 460 nm±15%, 480 nm±15%, 520 nm±15%, 550 nm±15%, 560 nm±15%, 606 nm±15%, 617 nm±15%, 620 nm±15%, 630 nm±15%, 650 nm±15%, 660 nm±15%, 705 nm±15%, 710 nm±15%, 720 nm±15%, 805 nm±15%, 810 nm±15%, 880 nm±15%, 905 nm±15%, 910 nm±15%, 950 nm±15%, 980 nm±15%, 980 nm±15%, 1050 nm±15%, 1200 nm±15%, 1310 nm±15%, 1380 nm±15%, 1450 nm±15%, 1600 nm±15%, 1800 nm±15%.

15. A device in accordance with claim 1, wherein at least the saturation of the blood with carbon monoxide SaCO can be determined in less than one minute and preferably less than 30 seconds by using at least two different wavelengths selected from the following group: 400 nm±15%, 460 nm±15%, 480 nm±15%, 520 nm±15%, 550 nm±15%, 560 nm±15%, 606 nm±15%, 617 nm±15%, 620 nm±15%, 630 nm±15%, 650 nm±15%, 660 nm±15%, 705 nm±15%, 710 nm±15%, 720 nm±15%, 805 nm±15%, 810 nm±15%, 880 nm±15%, 905 nm±15%, 910 nm±15%, 950 nm±15%, 980 nm±15%, 980 nm±15%, 950 nm±15%, 980 nm±15%, 980 nm±15%, 1050 nm±15%, 1200 nm±15%, 1310 nm±15%, 1380 nm±15%, 1450 nm±15%, 1600 nm±15%, 1800 nm±15%.

16. A device in accordance with claim 1, wherein physiological variables, such as, preferably, SaCO, SaO2, cHb, CaO2, or bilirubin, are determined and can be made available as information in such a way that at least two of the pV's can be made available as information on a display in a time interval of less than ten seconds.

17. A device in accordance with claim 1, wherein the device is portable.

18. A device in accordance with claim 1, wherein the device has a plastic housing (17) with a display (30), on which it is possible to display at least two pV's, which are selected from the following group: pulse, SaO2, SpO2, CaO2, SaCO, cHb, and MetHb, with operating buttons and a socket (23), which receives a sensor cable and is located in the housing, and with a compartment (28) for a power supply (26), which compartment (28) is located in the housing and is covered by a cover (25).

19. A device in accordance with claim 1, wherein the dimensions of the device of the invention are less than 15 cm in length, less than 8 cm in width, and less than 5 cm in depth.

20. A device in accordance with claim 1, wherein the volume of the device is less than 600 cm³.

21. A device in accordance with claim 1, wherein the device consists of no more than two printed circuit boards (20, 22) and fewer than 11 individual parts and/or fewer than three fasteners (27).

22. A device in accordance with claim 1, wherein the total weight of the device is less than 120 g and/or the housing has proportions of length to width to depth of about 3:2:1.

23. A device, which emits electromagnetic waves, especially light, of at least two different wavelengths and/or of at least two different bands of wavelengths from at least one source, where the electromagnetic waves are passed through a living and/or dead medium, preferably animal and/or human tissue and/or vessels to be tested, and the transmitted and/or reflected component of the electromagnetic waves is detected by a receiving system, which is suitable for detecting the light signals of different wavelengths in a time interval of less than one second, converting them to current and/or voltage signals that correspond to at least one measured value, and relaying them further, wherein at least one measured value from an evaluation unit is conditioned by signal conditioning, and, independently of the original wavelength, a measured value is further processed by active and/or passive electronic components.

24. A device for the determination of physiological variables (pV's) in tissue and/or vessels with
(a) at least one light source, which emits light, such that the light source is disposed in such a way that the light it emits is able to penetrate the tissue/vessel;
(b) at least one photodetector (PD), which is disposed in such a way that it detects the light that is emitted by the light sources and transmitted and/or backscattered by the tissue/vessel;
(c) an evaluation unit, which is connected with the photodetector (PD),
(d) such that, for at least one pV to be measured, the evaluation unit supplies a displayable output signal to an interface that can be connected to the evaluation unit,
(e) a socket and/or interface for receiving sensor signals, wherein
the device can determine at least two pV's selected from the following group: pulse, SaO2, CaO2, SaCO, and cHb.

25. A device in accordance with claim 1, wherein the device is realized as an original equipment manufacturer (OEM) printed circuit board, which can be mounted in patient monitors by at least one plug contact, which allows detachable mechanical and/or electronic coupling, and in this way it is possible, by simply adding the OEM printed circuit board of the invention, to expand the range of functions of patient monitors by the functions that are implemented on the OEM printed circuit board of the invention.
